SEQUENCE LISTING (13 sequences)

SEQ ID NO: 1 - protein, 81 aa, bovine
MEHWGEPIPRTGQSWRQPLSTSGRGWLGSAPSRWLGPASWRWLGPASWRWLGSAPWWWLGTATWWWRLGSRWYPRSMEQTQ

SEQ ID NO: 2 - protein, 64 aa, sheep
MEHWGEPIPGTGQSWRQPLPTSGRGWLGSAPWRWLGPTSWRWLGSAPWWWLGTATWWWRLGSRW

SEQ ID NO: 3 - protein, 65 aa, homo sapiens
MEHWGQPIPGAGQPWRQPLPTSGRWWLGAASWWWLGAASWWWLGAAPWWWLGSRRWHPQSVEQAE

SEQ ID NO: 4 - protein, 52 aa, mouse
MGAAGDNLMVVVGVSPMAVDGAKEGVPIISGTSPANQKPTSSIWQGLRQLGQ

SEQ ID NO: 5 - protein, 39 aa, hamster
MGTAPWWWLGTTSWWWLGSAPWWWLGSRRWHPQSVEQAQ

SEQ ID NO: 6 - protein, 16 aa, bovine
  UNSURE (1)..(16): Sequence of antigenic epitopes
RLGSRWYPRSMEEQTQ

SEQ ID NO: 7 - protein, 16 aa, sheep
PLPTSGRGWLGSAPWR

SEQ ID NO: 8 - protein, 14 aa, Homo sapiens
GSRRWHPQSVEQAE

SEQ ID NO: 9 - protein, 12 aa, mouse
RRWHPQSVEQAQ

SEQ ID NO: 10 - protein, 12 aa, hamster
SPMVDGAKEGVP

SEQ ID NO: 11 - protein, 22 aa, bovine
  DNA_BIND (1)..(22): sequence of DNA positive regulator of BSAS from 48 to 69 in the complete sequence ( See SEQ ID NO 1
WRWLGSAPWWWLGTATWWWRLG

SEQ ID NO: 12 - protein, 22 aa, sheep
  DNA_BIND (1)..(22): sequence of DNA positive regulator of SCRAPAS from 40 to 61 in the complete sequence (SEE SEQ ID NO 2
WRWLGSAPWWWLGTATWWWRLG

SEQ ID NO: 13 - protein, 28 aa, homo sapiens
  TRANSMEM (1)..(28): membrane spanning helix of CJAS from amino acid 25 to amino acid 52 in the complete sequenc
WWLGAASWWWLGAASWWWLGAAPWWWLG

(19) United States
(12) Patent Application Publication
(10) Pub. No.: US 2003/0022829 A1
(43) Pub. Date: Jan. 30, 2003

(54) Novel antiviral activities primate theta defensins and mammalian cathelicidins

(76) Inventors: Wendy Maury, Coralville, IA (US); Jack Stapleton, Iowa City, IA (US); Richard Roller, Coralville, IA (US); Mark Stinski, North Liberty, IA (US); Paul B. McCray, Iowa City, IA (US); Brian Tack, Iowa City, IA (US)

Correspondence Address: Steven L. Highlander, Fulbright & Jaworski L.L.P., Suite 2400, 600 Ccongress Avenue, Austin, TX, 78701, US

(21) Appl. No.: 10/060,102

(22) Filed: Jan. 29, 2002

Related U.S. Application Data
(60) Provisional application No. 60/265,270, filed on Jan. 30, 2001.
(60) Provisional application No. 60/309,368, filed on Aug. 1, 2001.

(51) Int. Cl. A61K 38/04; C07K 16/00; C07K 5/00; A01N 37/18; A61K 38/00; C07K 7/00; C07K 17/00

(52) U.S. Cl. 514/12; 530/326; 530/324; 514/2

(57) ABSTRACT

The present invention relates to the use of anti-viral peptides in the inhibition and treatment of viral infections, in particular infections caused by enveloped viruses. These anti-viral peptides, some natural and others artificial, adopt either amphiphilic alpha-helical or a theta structure where the homodimeric or heterodimer peptides are joined by both cysteine bonds and circularization of the peptides. These agents may be used alone or in combination with more traditional anti-viral pharmaceuticals.

This application claims benefit of the filing dates of U.S. Provisional Patent Application Ser. Nos. 60/265,270 and 60/309,368, filed on Jan. 30, 2001 and Aug. 1, 2001, respectively. The entire text of the above-referenced disclosure is specifically incorporated by reference herein in its entirety without disclaimer.

### BACKGROUND OF THE INVENTION

1. Field of the Invention

The present invention relates generally to the fields of molecular biology and virology. More particularly, it concerns the use of anti-viral peptides for the reduction of virus infectivity and treatment of viral infection.

2. Description of Related Art

Viral infections continue to be a major cause of disease in the world, with many causing significant mortalities, as well as contributing substantially to health care costs. For example, the epidemic of HIV in the underdeveloped world is both socially and economically devastating. The ongoing spread of HIV in regions of Africa and Asia is well documented. In these areas of the world, transmission between adults primarily occurs through heterosexual contact. Unfortunately, means of controlling sexual transmission of HIV are currently limited to barrier methods such as condoms that are not always culturally acceptable. The incorporation of viricidal compounds into a vaginal cream could potentially have profound effects on the worldwide spread of HIV. Currently, no such compounds are available. This also highlights the general lack of anti-viral drugs, as compared to the numerous anti-bacterial agents available.

Antimicrobial peptides have been isolated from plants, insects, fish, amphibia, birds, and mammals (Gallo, 1998; Ganz & Lehrer, 1998). Although previously considered an evolutionarily ancient system of immune protection with little relevance beyond minimal primary protection, recent developments have found that mammalian cells express these peptide antibiotics during inflammatory events such as wound repair, contact dermatitis and psoriasis (Nilsson, 1999). These peptides are apparently a primary component of innate host protection against microbial pathogenesis functioning to create pores in the cytoplasmic membrane of microorganisms (Oren et al., 1998). Furthermore, antimicrobial peptides also act on animal cells by stimulating them to change behaviors such as syndecan expression, chemotaxis, and chloride secretion (Gallo, 1998). After contact with microorganisms, vertebrate skin, trachea and tongue epithelia are rich sources of peptide antibiotics, which may explain the unexpected resistance of these tissues to infection (Russell et al. 1996).

There is no previous link between anti-microbial peptides and anti-viral activity. The ability to identify an anti-viral peptide would be a major advance in the treatment of viral diseases.

### SUMMARY OF THE INVENTION

The present invention provides new methods, combined compositions and kits, for use in inhibiting viral growth and proliferation, reducing viral burden and shed, inhibiting resistance to conventional anti-viral medications, and providing novel anti-virals for treating infections. The invention rests in the surprising use of one or more anti-viral peptides alone, or in conjunction with an anti-viral agent in the control of viral growth, proliferation, replication, or infection, and diseases arising therefrom.

The invention therefore encompasses methods, compositions, and kits that relate to an anti-viral peptide. The peptide may comprise natural or non-natural amino acids. It generally will be in the range of about 13 to about 35 amino acids, but includes peptides of specific lengths 13, 14, 15, 16, 17, 18, 19, 20, 21, 22, 23, 24, 25, 26, 27, 28, 29, 30, 31, 32, 33, 34 and 35 residues.

One embodiment thus represents a naturally-occurring anti-viral peptide selected from SEQ ID NOS: 1-7 (LL37, mCRAMP, Fall39, rCRAMP, SMAP29, SMAP28, and CAP 18) or a non-naturally-occurring peptide selected from the group consisting of SEQ ID NOS: 8 - 26 (OV-1, OV-2, OV-2.1, OV-2.2, OV-2.3, OV-3, OV-3.1, OV-3.2, OV-3.3, OV-4, OV-4.1, OV-4.2, OV-4.3, OV-5, OV-6, OV-7 and OV-8). Other anti-viral peptides of the present invention include human theta-defensins (SEQ ID NO: 27), rhesus monkey theta defensins (SEQ ID NOS: 28-30), chimeric human/rhesus monkey theta-defensins (SEQ ID NOS: 31-32). An additional embodiment would consist of a pharmaceutical composition wherein said composition comprises any of the aforementioned the anti-viral peptides and a pharmaceutically acceptable carrier.

In a further embodiment of the invention, an anti-viral peptide will be introduced into an environment, including but not limited to a host, in order to inhibit the growth and/or proliferation of viruses. Such an introduction envisions that the virus particle will be contacted by the anti-viral peptide, and as a result of this contact, the growth and or proliferation of the virus will be inhibited. Such a method may further consist of administering an anti-viral peptide in a pharmaceutically acceptable carrier and/or in combination with a second anti-viral agent. Such second anti-viral agents or antibiotics may include but are not limited to a naturally-occurring anti-viral peptide selected from SEQ ID NOS: 1-7 (LL37, mCRAMP, Fall39, rCRAMP, SMAP29, SMAP28, and CAP 18) or a non-naturally-occurring peptide selected from the group consisting of SEQ ID NOS: 8-26 (OV-1, OV-2, OV-2.1, OV-2.2, OV-2.3, OV-3, OV-3.1, OV-3.2, OV-3.3, OV-4, OV-4.1, OV-4.2, OV-4.3, OV-5, OV-6, OV-7 and OV-8), SEQ ID NOS: 27-32 or a protease inhibitor, a nucleoside analog, a viral polymerase inhibitor, and a viral integrase inhibitor.

An additional embodiment would consist of a method of inhibiting viral growth in a host, comprising administering to said host an anti-viral peptide selected from the group consisting of a naturally-occurring anti-viral peptide selected from SEQ ID NOS: 1-7 (LL37, mCRAMP, Fall39, rCRAMP, SMAP29, SMAP28, and CAP 18) or a non-naturally-occurring peptide selected from the group consisting of SEQ ID NOS: 8-26 (OV-1, OV-2, OV-2.1, OV-2.2, OV-2.3, OV-3, OV-3.1, OV-3.2, OV-3.3, OV-4, OV-4.1, OV-4.2, OV-4.3, OV-5, OV-6, OV-7 and OV-8) or SEQ ID NOS: 27-32.

The virus particle or population may be contacted either in vitro or in vivo. Contacting in vitro may further utilize mixture of fluids, including agitation such as rocking or repeated inversion. Contacting in vivo may be achieved by administering to an animal (including a human patient) that has or is suspected to have a viral infection, or is at risk of viral infection, a therapeutically effective amount of pharmacologically acceptable anti-viral peptide formulation alone or in combination with a therapeutic amount of a pharmacologically acceptable formulation of a second agent. The invention may thus be employed to treat both systemic and localized viral infections by introducing the agent or agents into the general circulation or by applying the combination, e.g., topically to a specific site.

An “effective amount of an anti-viral peptide” means an amount, or dose, within the range required to inhibit viral growth and/or proliferation, or to reduce the infectivity of a virus particle or population. Such ranges would be readily determinable by those of skill in the art depending upon the use to which the peptide is to be applied. An “effective amount of an anti-viral agent” means an amount, or dose, within the range normally given or prescribed. Such ranges are well established in routine clinical practice and will thus be readily determinable to those of skill in the art. Doses may be measured by total amount given or by concentration. Doses of 0.01, 0.05, 0.1, 0.5, 1, 2, 3, 4, 5, 6, 7, 8, 9, 10, 15, 20, 25, 30, 40, 50, 100, 500 and 1000 μg/ml solutions all are appropriate for treatment.

As this invention provides for enhanced viral killing, it will be appreciated that effective amounts of a second anti-viral agent may be used that are lower than the standard doses previously recommended, when the second anti-viral is combined with an anti-viral peptide. It is further envisioned that the anti-viral peptide may be used in combination with these other anti-viral agents for a variety of purposes. These purposes include but are not limited to enhancing the activity of the anti-viral agent, allowing for a lower dose of an anti-viral due to toxicity or dosing concerns relating to the second agent, enhancing the activity of anti-viral agents against strains that have previously exhibited resistance to an anti-viral agent, or providing an additional anti-viral agent in individuals whose immune system is damaged or compromised and are thus unable to mount an effective immune response.

Where a combination of an anti-viral peptide and one or more conventional anti-viral agents or antibiotics is contemplated, it is envisioned that the anti-viral peptide and the second anti-viral agent may be delivered either simultaneously or either of the agents may be administered prior to the administration of the other. It is envisioned that staggered administration might reduce the infectivity or number of viruses and increase the efficacy of the additional agent.

In a particular embodiment of the invention, an anti-viral peptide will be used alone or in combination with one or more additional anti-viral agents in the treatment of virus strains previously determined to be resistant to one or more methods of treatment. It is envisioned that this method will comprise inhibiting the growth of drug-resistant virus strains comprising administering to an environment capable of sustaining such growth an anti-viral peptide selected from the group consisting of a naturally-occurring anti-viral peptide selected from SEQ ID NOS: 1-7 (LL37, mCRAMP, Fall39, rCRAMP, SMAP29, SMAP28, and CAP 18) or a non-naturally-occurring peptide selected from the group consisting of SEQ ID NOS: 8-26 (OV-1, OV-2, OV-2.1, OV-2.2, OV-2.3, OV-3, OV-3.1, OV-3.2, OV-3.3, OV-4, OV-4.1, OV-4.2, OV-4.3, OV-5, OV-6, OV-7 and OV-8) and SEQ ID NOS: 27-32. Pharmaceutically acceptable compositions may be formulated such that resistant strains may be treated in a host either ex vivo or in vivo depending upon the requisite circumstances. In a particular embodiment, the anti-viral peptide is formulated for use intravaginally, for example, with a diaphragm or condom, optionally including a contraceptive (e.g., spermicidal, sperm immobilizing agent) composition.

A further embodiment of the invention envisions a nucleic acid molecule encoding the anti-viral peptide selected from the group consisting of a naturally-occurring anti-viral peptide selected from SEQ ID NOS: 1-7 (LL37, mCRAMP, Fall39, rCRAMP, SMAP29, SMAP28, and CAP 18) or a non-naturally-occurring peptide selected from the group consisting of SEQ ID NOS: 8-26 (OV-1, OV-2, OV-2.1, OV-2.2, OV-2.3, OV-3, OV-3.1, OV-3.2, OV-3.3, OV-4, OV-4.1, OV-4.2, OV-4.3, OV-5, OV-6, OV-7 and OV-8) and SEQ ID NOS: 27-32. It is envisioned that uses of these nucleic acid sequences could include, but are not limited to, creation of degenerate probes for the detection of further anti-viral peptide species, use in gene transfer or in the creation of fusion constructs linking the anti-viral peptides of the instant invention to other proteins.

A further embodiment consists of a kit for use in inhibiting viral growth in a host comprising an anti-viral peptide selected from the group consisting of a naturally-occurring anti-viral peptide selected from SEQ ID NOS: 1-7 (LL37, mCRAMP, Fall39, rCRAMP, SMAP29, SMAP28, and CAP 18) or a non-naturally-occurring peptide selected from the group consisting of SEQ ID NOS: 8-26 (OV-1, OV-2, OV-2.1, OV-2.2, OV-2.3, OV-3, OV-3.1, OV-3.2, OV-3.3, OV-4, OV-4.1, OV-4.2, OV-4.3, OV-5, OV-6, OV-7 and OV-8) and SEQ ID NOS: 27-32, in a suitable container. In an additional embodiment, a kit may contain the anti-viral peptide and a second anti-viral agent. The second anti-viral agent may be selected from the group consisting of a protease inhibitor, a nucleoside analog, a viral polymerase inhibitor, and a viral integrase inhibitor.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C—Ability of Ov-1 to inhibit virus replication. FIG. 1A. Inhibition of HSV plaque formation by increasing concentrations of Ov-1 and the parental peptide, SMAP 29. Triangles=HSV1 with SMAP29; diamonds=HSV2 with SMAP29; open circles=HSV1 with Ov-1; squares=HSV2 with Ov-1. FIG. 1B. Inhibition of EIAV infectious titers by increasing concentrations of Ov-1. FIG. 1C. Inhibition of HIV infectious titers by increasing concentrations of Ov-1. All virus was preincubated with the appropriate concentration of peptide and mixture was added to cells. HSV plaques were scored 18 h post infection. Lentiviral immunostaining assays were performed on fixed cells 40-44 h post infection.

FIGS. 2A-2D—Inhibition of CMV cytopathology by Ov-1. FIG. 2A. Uninfected monolayer of primary human fibroblasts. FIGS. 2B-D. Primary human fibroblasts infected with CMV at a MOI of approximately 5. FIG. 2B. No peptide added, but 37° C. preincubation of virus performed. FIG. 2C. 5 μg/ml Ov-1 incubated with virus for 1 h at 37° C. prior to addition to monolayer. FIG. 2D. Twenty μg/ml Ov-1 incubated with virus for 1 h at 37° C. prior to addition to monolayer. Media was changed on all monolayers 3 days, post infection. Cells were fixed and stained at 14 days post infection. Plaque formation and accompanying monolayer disintegration can be observed in FIGS. 2B and 2C. No plaque formation was detected when virus was incubated with 20 μg/ml of Ov-1.

FIGS. 3A and 3B—Ability of the Ov series to inhibit expression of lentiviruses. FIG. 3A. Ability of 10 μg/ml of Ov-1 and the amino terminal peptides to inhibit

EIAV expression at 40 h, post infection. The MA-1 strain of EIAV was used in the equine dermal cell line, ED, for these studies. T7 and G10 represent Ov-2(18T7) and Ov-2(18G10), respectively. Horse anti-EIAV antisera (1:800) was used to immunodetect EIAV-infected cells. FIG. 3B. Ability of 8 μg/ml of Ov-1 and the carboxyl terminal peptides to inhibit HIV expression at 40 h post infection. The pNL4-3 strain of HIV was used in HeLa 37 cells for these studies. Human anti-HIV capsid mAb (1:150) was used to immunodetect HIV-infected cells.

FIGS. 4A-4D—Ability of theta defensins to inhibit HIV-1 replication. FIG. 4A. Antiviral activity of theta defensins against HIV-1. Infected cultures were immunostained for HIV infection 40 h post infection. Oxidized (ox) and oxidized, circularized (dcc) forms of human theta defensin-1 (HTD-1) and rhesus theta defensin-3 (RTD-3) were preincubated for 15 minutes prior to adding the mixture to the cells. FIG. 4B. Inhibition curve of increasing concentrations of oxidized and oxidized, circularized HTD-1 on HIV replication in HeLa cells. FIGS. 4C & 4D. Logistic dose response curve plots of HIV inhibition by HTD-1 ox and HTD-1 dcc. IC₅₀s were determined from these plots.

### DESCRIPTION OF ILLUSTRATIVE EMBODIMENTS

Although numerous antibiotic agents are available for the treatment of bacterial and even fungal infections, relatively few drugs are available for the use of viral infections. Yet each year, millions of people are infected with viruses that range from the relatively innocuous (e.g., rhinoviruses) to those that are quite deadly (e.g., HIV). Therefore, in order to maintain the present standards of public health and to limit growing health care costs, new methods of controlling viral infection must be devised.

Antimicrobial peptides of higher eukaryotes, though long recognized as components of the innate immune system, were initially considered primitive and of little clinical significance. However, the relative simplicity of these peptides belies their importance, not only in the prevention of primary microbial infection, but also in subsequent immunomodulation. Further, the small size of the molecules suggests a decreased sensitivity to many of the mechanisms of microbial resistance. Antimicrobial peptides are generally lethal to bacteria and some fungi. They exhibit differential toxicity towards mammalian cells (Hwang et al., 1998). While the mechanism of this action is not definitively known, it is believed that the peptides interact with the lipid bilayer and may thus compromise the integrity of the bacterial membrane (Hwang et al., 1998).

Cathelidicins are a diverse group of naturally occurring, cationic peptides with strong antimicrobial activity. The inventors explored the antiviral activity of a number of these peptides. In work described herein, two members of this group were found to significantly inhibit the replication of some enveloped viruses. mCramp, a murine cathelicidin, reduced herpes simplex 1 and 2 replication by approximately 75%, and consistently reduced the infectivity of two retroviruses, human immunodeficiency virus (HIV) and equine infectious anemia virus (EIAV). Similar concentrations of mCramp had no inhibitory effect on vaccinia virus replication. OV-1 is a synthetic peptide modeled on the sheep cathelicidin Smap29. OV-1 had the strongest antiviral activity of the peptides tested inhibiting both the herpes viruses and retroviruses at an effective LD₅₀ of approximately 3 μg/ml. The observed inhibition of these diverse enveloped viruses suggested that OV-1 may be acting at the viral envelope. This hypothesis is consistent with previous bacterial studies which have demonstrated that cathelicidins disrupt bacterial membrane integrity.

To assess the region(s) of OV-1 which confer its ability to inhibit enveloped virus infectivity, a shortened form was tested for antiviral activity. OV-2.3 is composed of the 18 amino-terminal residues of OV-1. NMR studies of OV-2.3 have demonstrated that it retains the α-helical structure of OV-1 in membrane mimetic environments. Significant levels of viral replication inhibition were detected with OV-2.3. From these studies, the inventors have determined that the α-helical conformation of OV-2.3 is of a sufficient length to span the lipid bilayer, although shorter peptides also may suffice.

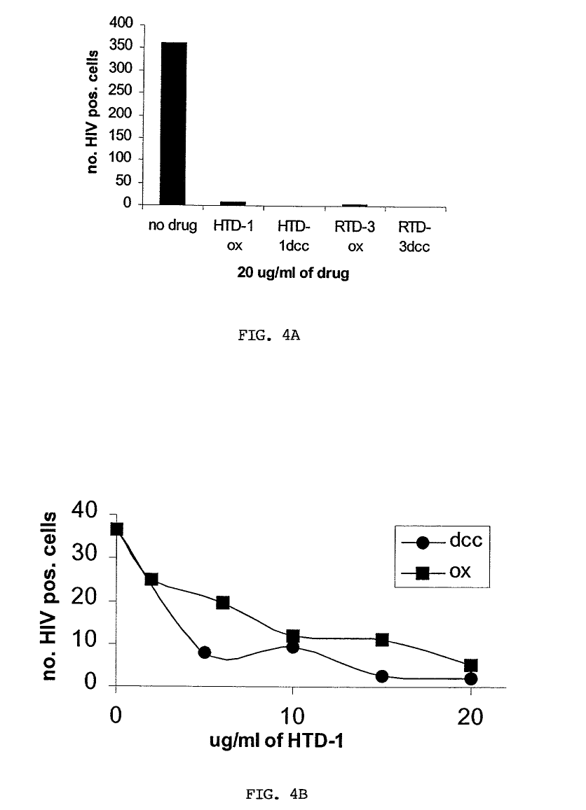

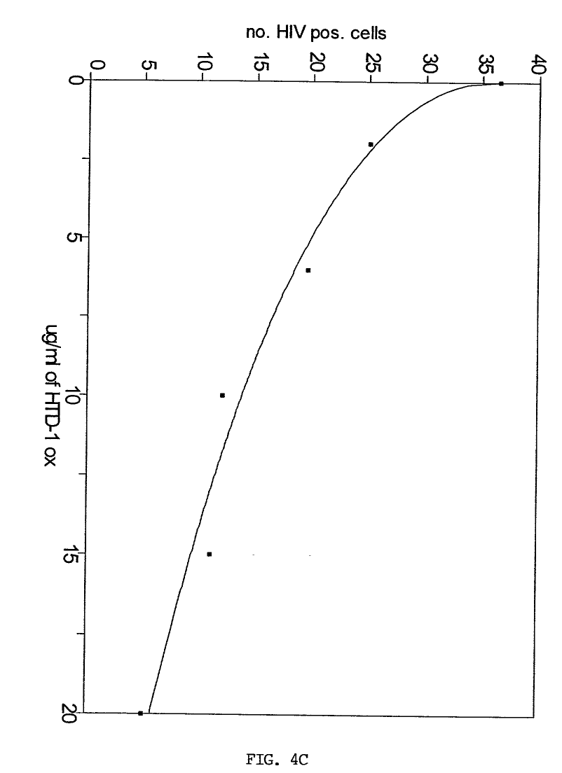

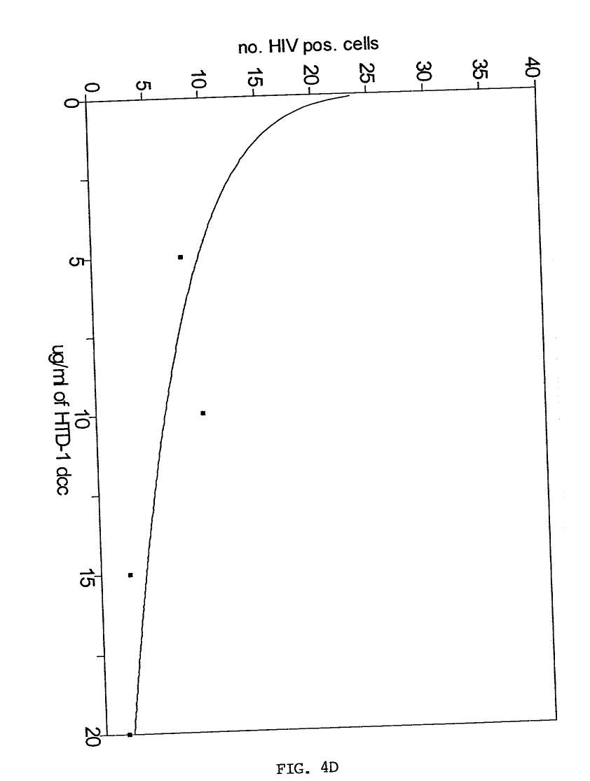

A series of synthetically derived theta defensins from rhesus macaques (RTD 1-3) have been shown to exhibit bactericidal activity (Tang, 1999; Tran, 2001). These compounds were tested for anti-viral activity. In addition to the RTD peptides, synthetic peptides specified from human pseudogene, human theta defensin 1, was tested for anti-viral activity. Both oxidized and oxidized circular forms of the homodimeric and heterodimeric peptides were investigated. As shown in FIGS. 4A-D, theta defensins effectively inhibited HIV replication in HeLa cells. Both the oxidized and oxidized, circular forms of human theta defensin-1 (SEQ ID NO: 27) and rhesus theta defensin-3 (SEQ ID NO: 29) were most effective in blocking acute HIV replication as determined in the 40 h HIV infectivity described above. The oxidized, circularized forms of the theta peptides consistently were most effective at blocking HIV than the oxidized forms. IC₅₀ values determined in a dose response curve indicated that oxidized HTD-1 inhibited HIV replication with an IC₅₀ of approximately 4.5 ug/ml whereas the approximate IC₅₀ of oxidized, circularized HTD-1 was 0.45 ug/ml.

Thus, this invention encompasses methods to inhibit viral infection through the use of synthetic peptides. It is contemplated that these peptides may be delivered into an environment in which viruses are present or are likely to be present in order to control their growth and proliferation. It is further envisioned that such an environment would include a host organism. These embodiments, as well as others, are set forth in the following detailed description of the invention.

### I. Anti-Viral Peptide, Peptide Production, Purification and Delivery

### A. Antiviral Microbial Peptides

As discussed above, a number of different organisms have been identified as producing antimicrobial peptides—humans, mice, sheep, monkeys for example. Human beta-defensins, human and monkey theta defensins (and chimeric structures thereof) and cathelicidins are therefore included within the scope of the present invention. Both natural and synthetic variants of these molecules are provided, and illustrated in the following tables.

TABLE 1
Natural Anti-Viral Peptides

| Peptide | Peptide Sequence | SEQ ID NO |
|---|---|---|
| mCRAMP | ISRLAGLLRKGGEKIGEKLKKIGQKIKNFFQKLVPQPEQ | SEQ ID NO:1 |
| rCRAMP | ISRLAGLVRKGGEKFGEKLRKIGQKIKEFFQKLALEIEQ | SEQ ID NO:2 |
| SMAP28 | RGLRRLGRKIAHGVKKYGPTVLRIIRIA-NH2 | SEQ ID NO:3 |
| SMAP29 | RGLRRLGRKIAHGVKKYGPTVLRIIRIAG | SEQ ID NO:4 |
| CAP18 | GLRKRLRKFRNKIKEKLKKIGQKIQGLLPKLAPRTDY | SEQ ID NO:5 |
| FALL39 | FALLGDFFRKSKEKIGKEFKRIVQRIKDFLRNLVPRTES | SEQ ID NO:6 |
| LL37 | LLGDFFRKSKEKIGKEFKRIVQRIKDFLRNLVPRTES | SEQ ID NO:7 |

TABLE 2
Synthetic Anti-Viral Peptides (Ovispirins)

| Peptide | Sequence | SEQ ID NO |
|---|---|---|
| Ovispirin 1 | KNLRRIIRKIIHIIKKYGPTILRIIRIIG-NH2 | SEQ ID NO:8 |
| (OV-1) | | |
| OV-2.3 | KNLRRIIRKIIHIIKKYG-NH2 | SEQ ID NO:9 |
| OV-2.2 | KNIRRIIRKIIHIIKKYG-NH2 | SEQ ID NO:10 |
| OV-2.1 | KNIRRIIRKIIHIIKKYG | SEQ ID NO:11 |
| OV-2 | KNLRRIIRKIIHIIKKYG | SEQ ID NO:12 |
| OV-3 | LRRIIRKIIHIIKK-NH2 | SEQ ID NO:13 |
| OV-3.1 | NLRRIIRKIIHIIKKY | SEQ ID NO:14 |
| OV-3.2 | NIRRIIRKIIHIIKKY | SEQ ID NO:15 |
| OV-3.3 | Ac-KIIHIIKKYGPTILRIIRIIG-NH2 | SEQ ID NO:16 |
| OV-4 | KIIHIIKKYGPTILRIIRIIG-NH2 | SEQ ID NO:17 |
| OV-4.1 | LRRIIRKIIHIIKK | SEQ ID NO:18 |
| OV-4.2 | IRRIIRKIIHIIKK-NH2 | SEQ ID NO:19 |
| OV-4.3 | IRRIIRKIIHIIKK | SEQ ID NO:20 |
| OV-5 | IHIIKKYGPTILRIIRIIG-NH2 | SEQ ID NO:21 |
| OV-6 | HIIKKYGPTILRIIRIIG-NH2 | SEQ ID NO:22 |
| OV-7 | Ac-IHIIKKYGPTILRIIRIIG-NH2 | SEQ ID NO:23 |
| OV-8 | Ac-HIIKKYGPTILRIIRIIG-NH2 | SEQ ID NO:24 |
| Ov-2(T7) | KNLRRITRKIIHIIKKYG | SEQ ID NO:25 |
| Ov-2(G10) | KNLRRIIRKGIHIIKKYG | SEQ ID NO:26 |
| HTD-1 | GICRCICGRGICRCICGR | SEQ ID NO:27 |
| RTD-2 | GFCRCICTRGFCRCICTR | SEQ ID NO:28 |
| RTD-3 | GVCRCLCRRGVCRCLCRR | SEQ ID NO:29 |
| RTD-1 | GFCRCLCRRGVCRCICTR | SEQ ID NO:30 |
| H/RTD-3 | GICRCLCRRGVCRCICGR | SEQ ID NO:31 |
| H/RTD-2 | GICRCICTRGFCRCICGR | SEQ ID NO:32 |

### B. Peptide Synthesis

The anti-viral peptides envisioned in the present embodiment of the invention may be chemically synthesized. An example of a method for chemical synthesis of such a peptide is as follows. Using the solid phase peptide synthesis method of Sheppard et al. (1981) an automated peptide synthesizer (Pharmacia LKB Biotechnology Co., LKB Biotynk 4170) adds N,N′-dicyclohexylcarbodiimide to amino acids whose amine functional groups are protected by 9-fluorenylmethoxycarbonyl groups, producing anhydrides of the desired amino acid (Fmoc-amino acids). An Fmoc amino acid corresponding to the C-terminal amino acid of the desired peptide is affixed to Ultrosyn A resin (Pharmacia LKB Biotechnology Co.) through its carboxyl group, using dimethylaminopyridine as a catalyst. The resin is then washed with dimethylformamide containing iperidine resulting in the removal of the protective amine group of the C-terminal amino acid. A Fmoc-amino acid anhydride corresponding to the next residue in the peptide sequence is then added to the substrate and allowed to couple with the unprotected amino acid affixed to the resin. The protective amine group is subsequently removed from the second amino acid and the above process is repeated with additional residues added to the peptide in a like manner until the sequence is completed. After the peptide is completed, the protective groups, other than the acetoamidomethyl group are removed and the peptide is released from the resin with a solvent consisting of, for example, 94% (by weight) trifluroacetic acid, 5% phenol, and 1% ethanol. The synthesized peptide is subsequently purified using high-performance liquid chromatography or other peptide purification technique discussed below.

The homodimeric and heterodimer and chimeric forms of RTDs and HTD-1 were synthesized (Tang et al., 1999). A volume of 10% DMSO was included in the oxidation step that facilitated the reaction and improved yields of the oxidized form. In addition, dicyclohexylcarbodiimide (dcc) was employed for circularization or ring closure rather than carbodiimide. The oxidized and oxidized, circularized peptides were subsequently purified using high performance liquid chromatography as described below.

In designing alternate peptide constructs with enhanced anti-viral properties, substitutions may be used which modulate one or more properties of the molecule. Such variants typically contain the exchange of one amino acid for another at one or more sites within the peptide. For example, certain amino acids may be substituted for other amino acids in a peptide structure in order to enhance the interactive binding capacity of the structures. Since it is the interactive capacity and nature of a protein that defines that protein's biological functional activity, certain amino acid substitutions can be made in a protein sequence (or its underlying DNA coding sequence) which potentially create a peptide with superior characteristics. In particular, those changes that enhance the amphipathic, α-helical nature will be most desired.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules.

Each amino acid has been assigned a hydropathic index on the basis of their hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

Amino acid substitutions are generally based on the relative similarity of the amino acid side-chain substituents, for example, their hydrophobicity, hydrophilicity, charge, size, and the like but may nevertheless be made to highlight a particular property of the peptide. Exemplary substitutions that take the foregoing characteristics into consideration are well known to those of skill in the art and include: arginine and lysine; glutamate and aspartate; serine and threonine; glutamine and asparagine; and valine, leucine and isoleucine.

It also is possible to create anti-viral peptides by genetic means, ie., cloning and expression. In particular, it is envisioned that the constructions of fusion proteins will involve fusion of a nucleic acid sequence encoding the anti-viral peptide with a cDNA encoding the desired fusion partner, followed by recombinant expression. The anti-viral peptide sequences disclosed in this application are readily created from artificial or natural DNAs. Such sequences may be prepared synthetically, but also through conventional techniques using probes to recover corresponding DNAs from genomic or cDNA libraries. Following cloning, such DNAs can then be incorporated in appropriate expression vectors and used to transform host cells (e.g., bacterial or mammalian cells), which can be cultured to form recombinant anti-viral peptides.

As used in this application, the tern “an isolated nucleic acid encoding an anti-viral peptide refers to a nucleic acid molecule that has been isolated free of total cellular nucleic acid. The term “functionally equivalent codon” is used herein to refer to codons that encode the same amino acid, such as the six codons for arginine or serine (Table 3), and also refers to codons that encode biologically equivalent amino acids, as discussed in the following pages.

TABLE 3
Codons

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The DNA segments of the present invention include those encoding biologically functional equivalent antimicrobial peptides, as described above. Functionally equivalent proteins or peptides may be created via the application of recombinant DNA technology, in which changes in the protein structure may be engineered, based on considerations of the properties of the amino acids being exchanged, or as a result of natural selection. Changes designed by man may be introduced through the application of site-directed mutagenesis techniques or may be introduced randomly and screened later for the desired function.

Also encompassed within the term “proteinaceous composition” are proteins that include at least one modified or unusual amino acid, including but not limited to those shown on Table 4 below.

TABLE 4
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3- Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4- Aminobutyric acid, piperidinic acid | 4Hyp | 4-Hydroxyproline |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

### C. Fusion Proteins

As discussed above, the anti-viral peptides of the instant application may be combined with fusion partners to produce fusion proteins. It is envisioned that such constructs might include combinations of an anti-viral peptide with a partner also exhibiting some level of anti-viral activity. Such a construct generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification if such removal is desired. Other useful fusions include linking of functional domains, such as active sites from enzymes, glycosylation domains, cellular targeting signals or transmembrane regions.

### D. Expression of Anti-Viral Peptides

In other embodiments, it is envisioned that anti-viral peptides may be utilized in gene therapy. Individuals who are immunodeficient due to disease, injury or genetic defect may be the subject of gene therapy in which the genes for antimicrobial peptides are incorporated into host cells. To facilitate gene transfer, the cDNA for anti-viral peptides must be incorporated into an expression construct.

Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, the like.

### i. Regulatory Elements

Throughout this application, the term “expression construct” is meant to include any type of genetic construct containing a polynucleotide coding for a gene product in which part or all of the nucleic acid encoding sequence is capable of being transcribed. The transcript may be translated into a protein, but it need not be. In certain embodiments, expression includes both transcription of a gene and translation of MRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term eukaryotic promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of direction the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, adenovirus E1A promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

### ii. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

### iii. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together each separated by an IRES, creating polycistronic messages. By virtue of the IRES element each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

### iv. Host Cells and Delivery of Expression Vectors

Certain examples of prokaryotic hosts are E. coli strain RR1, E. coli LE392, E. coli B, E. coli X 1776 (ATCC No. 31537) as well as E. coli W3110 (F-, lambda-,prototrophic, ATCC No. 273325); bacilli such as Bacillus subtilis; and other enterobacteriaceae such as Salmonella typhimurium, Serratia marcescens, and various Pseudomonas species.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material, but can be readily introduced in a variety of cell lines and laboratory animals (Nicolas and Rubenstein, 1988; Temin, 1986).

One possible method for in vivo delivery involves the use of a virus that is not affected by the peptides of the invention—adenovirus expression vector has been shown to have minimal susceptibility, possibly because it does not utilize an envelope. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus & Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′ -tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1 or the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) discloses improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 hours. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 hours.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

In order to effect expression of gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990), DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al, 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a particular embodiment, liposomal formulations are contemplated. Liposomal encapsulation of pharmaceutical agents prolongs their half-lives when compared to conventional drug delivery systems. Because larger quantities can be protectively packaged, this allows the opportunity for dose-intensity of agents so delivered to cells. This would be particularly attractive in the chemotherapy of cervical cancer if there were mechanisms to specifically enhance the cellular targeting of such liposomes to these cells.

“Liposome” is a generic term encompassing a variety of single and multilamellar lipid vehicles formed by the generation of enclosed lipid bilayers. Phospholipids are used for preparing the liposomes according to the present invention and can carry a net positive charge, a net negative charge or are neutral. Dicetyl phosphate can be employed to confer a negative charge on the liposomes, and stearylamine can be used to confer a positive charge on the liposomes. Liposomes are characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are cationic lipid-nucleic acid complexes, such as lipofectamine-nucleic acid complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1987). In yet further embodiments, the liposome may be complexed or employed in conjunction with both HVJ and HMG-1. In that such expression vectors have been successfully employed in transfer and expression of a polynucleotide in vitro and in vivo, then they are applicable for the present invention. Where a bacterial promoter is employed in the DNA construct, it also will be desirable to include within the liposome an appropriate bacterial polymerase.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform, chloroform/methanol or t-butanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules will form a bilayer, known as a lamella, of the arrangement XY-YX.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis (1979), the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be reconstituted in a solution of nucleic acid and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentration and stored at 4° C. until use.

In a preferred embodiment, the lipid dioleoylphosphatidylcholine is employed. Nuclease-resistant oligonucleotides were mixed with lipids in the presence of excess t-butanol. The mixture was vortexed before being frozen in an acetone/dry ice bath. The frozen mixture was lyophilized and hydrated with Hepes-buffered saline (1 mM Hepes, 10 mM NaCl, pH 7.5) overnight, and then the liposomes were sonicated in a bath type sonicator for 10 to 15 min. The size of the liposomal-oligonucleotides typically ranged between 200-300 nm in diameter as determined by the submicron particle sizer autodilute model 370 (Nicomp, Santa Barbara, Calif.).

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand in combination with a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### E. Preparations

It is envisioned that the anti-viral peptides and any second agents that might be delivered may be formulated and administered in any pharmacologically acceptable vehicle, such as parenteral, topical, aerosal, liposomal, nasal or ophthalmic preparations, with formulations designed for oral administration being currently preferred due to their ease of use. It is further envisioned that formulations such as antimicrobial peptides and any second agents that might be delivered may be formulated and administered in a manner that does not require that they be coupled with a pharmaceutically acceptable carrier. In those situations, it would be clear to one of ordinary skill in the art the types of diluents that would be proper for the proposed use of the peptides and any secondary agents required. Although further purification following synthesis may be desired, it is not necessarily required for use.

In another embodiment, the anti-viral peptides may be used as a decontaminating agent. For example, they may be spray in a liquid or powdered form onto a surface or area that has contacted, or may come into contact with, a virus particle. This may have particular relevance to use in epidemics where rooms, buildings or outdoor areas may be treated. Similarly, if viruses are used as a biological warfare agent, equipment and troops may be treated by spraying, immersion, or swabbing. In addition, it also is possible to coat surfaces (e.g., protective suits or coverings, medical instruments) with peptides of the present invention.

### F. Protein Purification

Peptide purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic, immunologic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or HPLC.

Certain aspects of the present invention concern the purification, and in particular embodiments, the substantial purification, of an encoded peptide. The term “purified peptide” as used herein, is intended to refer to a composition, isolatable from other components, wherein the peptide is purified to any degree relative to its naturally-obtainable state. A purified peptide therefore also refers to a peptide, free from the environment in which it may naturally occur.

Generally, “purified” will refer to a peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more peptides in the composition. The term “purified to homogeneity” is used to mean that the composition has been purified such that there is single protein species based on the particular test of purity employed for example SDS-PAGE or HPLC.

Various methods for quantifying the degree of purification of the peptide will be known to those of skill in the art in light of the present disclosure. These include, for example, assessing the amount of peptides within a fraction by SDS/PAGE analysis.

There is no general requirement that the peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

It is known that the migration of a peptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffmity chromatography. The generation of antibodies that would be suitable for use in accord with the present invention is discussed below.

### II. Therapeutic Uses

This invention encompasses methods to reduce virus growth, infectivity, burden, shed, development of anti-viral resistance, and to enhance the efficacy of traditional anti-viral therapies. An attractive feature of these peptides is their tolerance for high salt concentrations. The peptides maintain activity in physiological salt solutions.

The anti-viral properties of the peptides disclosed in combination with their stability and insensitivity to high salt concentrations allow them to be included in formulations to inhibit virus growth and proliferation. The purified anti-viral peptides may be used without further modifications or they may be diluted in a pharmaceutically acceptable carrier. Because of the stability of the peptides, it is contemplated that the invention may be administered to humans or animals, included in food and pharmaceutical preparations. In addition, as stated above, they may also be used in medicinal and pharmaceutical products (such as fluid containers, i.v. bags, tubing, syringes, etc.), as well as in cosmetic products, hygenic products, cleaning products and cleaning agents, as well as any material to which the peptides could be sprayed on or adhered to wherein the inhibition of virucidal growth on such a material is desired.

The proper dosage of an anti-viral peptide necessary to prevent viral growth and proliferation depends upon a number of factors including the types of virus that might be present, the environment into which the peptide is being introduced, and the time that the peptide is envisioned to remain in a given area.

In particular, the invention is believed most applicable to enveloped viruses. For example, the Togoviridae, Flaviviridae, Coronoviridae, Rhabdoviridae, Filoviridae, Paramyxoviridae, Orthomyxoviridae, Bunyaviridae, Arenaviridae, Retroviridae, Herpesviridae, Poxviridae and Iridoviridae all should be susceptible to attack by the anti-viral peptides of the present invention.

Specific viruses include the human viruses HIV, HSV-1, HSV-2, EBV, CMV, herpesvirus B, HHV6, varicella zoster virus, HHV8, respiratory syncytial virus (RSV), influenza A, B and C viruses, hepatitis A, hepatitis B, hepatitis C, hepatitis G, smallpox, vaccinia virus, Marburg virus, ebola virus, dengue virus, West Nile virus, hantavirus, measles virus, mumps virus, rubella virus, rabies virus, yellow fever virus, Japanese encephalitis virus, Murray Valley encephalitis virus, Rocio virus, tick-borne encephalitis virus, St. Louis encephalitis virus, chikungynya virus, o'nyong-nyong virus, Ross River virus, Mayaro virus, human coronaviruses 229-E and OC43, vesicular stomatitis virus, sandfly fever virus, Rift Valley River virus, Lasa virus, lymphocytic choriomeningitis virus, Machupo virus, Junin virus, HTLV-I and -II. Other animal viruses include those of swine (swinepox, African swine fever virus, hemagluttinating virus of swine, hog cholera virus, pseudorabies virus), sheep (border disease virus, Maedi virus, visna virus), cattle (bovine leukemia virus, bovine diarrhea virus, bovine lentivirus, infectious bovine rhinotracheitis virus), horses (eastern and western equine encephalitis virus, Venezuelan equine encephalitis virus, equine infectious anemia virus, equine arteritis virus), cats (feline immunodeficiency virus, feline leukemia virus, feline infectious peritonitis virus), monkeys (simian hemorrhagic fever virus) and fowl (Marek's disease virus, turkey bluecomb virus, infectious bronchitis virus of fowl, avian reticuloendotheliosis, sarcoma, and leukemia viruses).

It is further contemplated that the anti-viral peptides of the invention may be used in combination with or to enhance the activity of other anti-viral agents. Combinations of the peptide with other agents may be useful to allow agents to be used at lower doses due to toxicity concerns, to enhance the activity of agents whose efficacy has been reduced or to effectuate a synergism between the components such that the combination is more effective than the sum of the efficacy of either component independently. Anti-virals which may be combined with an anti-viral peptide in combination therapy include but are not limited to a protease inhibitor, a nucleoside analog, a viral polymerase inhibitor, and a viral integrase inhibitor.

The phrases “pharmaceutically” or “pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. In particular, use of the anti-viral peptides of the present invention in a condom or diaphragm, optionally in conjunction with a spermicidal or other contraceptive substance, is envisioned. Alternatively, administration may be by orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and anti-fungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. Routes of administration may be selected from intravenous, intrarterial, intrabuccal, intraperitoneal, intramuscular, subcutaneous, oral, topical, rectal, vaginal, nasal and intraocular.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The purified anti-viral peptide may be used without further modifications or it may be diluted in a pharmaceutically acceptable carrier. The peptides may be used independently or in combination with other anti-viral or antimicrobial agents. Because of the stability of the peptides it is contemplated that the invention may be administered to humans or animals. It may also be included in food preparations, pharmaceutical preparations, medicinal and pharmaceutical products, cosmetic products, hygienic products, cleaning products and cleaning agents, as well as any material to which the peptides could be sprayed on or adhered to wherein the inhibition of viral growth is desired.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Peptide Synthesis

All peptides were synthesized by the solid-phase method employing an Applied Biosystems model 433A peptide synthesizer and Fastmoc strategy at the 0.1 mM scale. Peptides were purified by reversed-phase HPLC on a Waters Delta Prep employing a Vydac 218TP1022 (22×250 mm) column. Separation was performed with a gradient system of aqueous 0.1% trifluoroacetic acid (solvent A) and 100% acetonitrile containing 0.085% trifluoroacetic acid (solvent B). A linear gradient from 0 to 100% B was applied over 70 min and fractions collected every 0.2 min. Fractions were subsequently monitored by analytical scale reversed-phase HPLC on a Beckman Gold System using a Vydac 218TP54 (4.6×250 mm) column at a flow rate of 0.5 mmin under isocratic elution conditions. Select fractions were pooled and lyophilized; further characterization of peptides was provided by mass spectrometry and capillary electrophoresis. Mass measurements were performed by flow injection at 0.1 mmin in 64% acetonitrile containing 0.05% trifluoroacetic acid with a Hewlett-Packard model 1100 MSD equipped with an electrospray ionization source. Capillary electrophoresis was performed on a Hewlett-Packard 3D instrument equipped with an extended light path fused-silicate column 75 micrometers (ID)×80.5 centimeters (total length). Capillary electrophoresis experiments were conducted at 18° C. in 100 mM sodium phosphate buffer, pH 2.9 at 20,000 volts. Peptide concentration was determined by quantitative amino acid analysis on a Beckman 6300 Amino Acid analyzer.

### EXAMPLE 2

### Peptide Reagents

A series of synthetic peptides collectively called ovispirins were developed to assess the respective contributions of length, amphipathicity and α-helical content of the peptide to antimicrobial and anti-viral activity. These peptides were modeled from a naturally occurring, sheep cathelicidin-derived peptide, SMAP29 (SEQ ID NO: 4). One such peptide is a 29-mer called Ovispirin 1 (SEQ ID NO: 8, Ov-1). This synthetic peptide is predicted to have a strong amphipathic, α-helical structure in a lipid environment resulting from alterative capping of the helix and repeated isoleucine substitutions for less hydrophobic residues (Table 5).

In addition to the synthesis of SEQ ID NO: 8 (Ov-1), a series of smaller peptides derived from either the amino- or carboxy-termini of Ov-1 have also been synthesized.

Four amino-terminal forms have been generated called Ov-2(SEQ ID NO: 12, 18-mer), Ov-2 (SEQ ID NO: 25, T7), Ov-2 (SEQ ID NO: 26, G10) and Ov-3 (SEQ ID NO: 13, 14-mer). The Ov-2 series are 18-mers representative of the amino-terminal sequence of SEQ ID NO: 8. The peptides of SEQ ID NO: 25 and SEQ ID NO: 26 have amino acid substitutions of threonine for isoleucine at position 7 and glycine for isoleucine at position 10, respectively, that disrupt the amphipathic α-helical nature of the peptides. The peptides of SEQ ID NO: 25 and SEQ ID NO: 26 were kindly provided by Alan Waring and Robert Lehrer (Dept. of Medicine, UCLA). Ov-3 (14) (SEQ ID NO: 13) consists of the 14 amino-terminal amino acids of Ov-1. Peptides from the carboxy-terminal sequence of Ov-1 have also been synthesized. These include two 21-mers (The peptides of SEQ ID NO: 16,17; Ov-3.3 and Ov-4), two 19-mers (The peptides of SEQ ID NO: 21,23; Ov-5 and Ov-7) and two 18-mers (The peptides of SEQ ID NO: 22,24; Ov-6 and Ov-8). While the carboxyl-terminal peptides have not been studied for their anti-microbial activity, Ov-1 through Ov-3 have been studied and found to have potent anti-bacterial activity at concentrations of 0.5 to 8 μg/ml against a panel of respiratory pathogens.

TABLE 5
Physical and chemical characteristics of naturally occurring
sheep peptide, SMAP29, and its synthetic derivatives

| | SEQ | | Net | |
|---|---|---|---|---|
| | ID | | positive | % helicity in |
| Name | NO | Peptide Amino acid seq. | Charge | Phos. Buffer* |
| SMAP29 | 4 | RGLRRLGRKIAHGVKKYGPTVLRIIRIAG | 9 | 7.6 (57) |
| Ov-1(29) | 8 | KNLRRIIRKIIHIIKKYGPTILRIIRIIG-NH2 | 10 | 43.9 (97.9) |
| Ov-2(18) | 12 | KNLRRIIRKIIHIIKKYG | 8 | 27.2 (99.2) |
| Ov-2(T7) | 25 | KNLRRITRKIIHIIKKYG | 7 | 8.0 (66.8) |
| Ov-2(G10) | 26 | KNLRRIIRKGIHIIKKYG | 7 | 7.4 (50.4) |
| Ov-3 | 13 | LRRIIRKIIHIIKK-NH2 | 7 | 14.7 (66.3) |
| Ov-4 | 17 | KIIHIIKKYGPTILRIIRIIG | 5 | N/D |
| Ov-5 | 21 | IHIIKKYGPTILRIIRIIG | 4 | N/D |
| Ov-6 | 22 | HIIKKYGPTILRIIRIIG | 4 | N/D |

*parenthetical indicates % helicity in phosphate buffer + 40% TFE

Furthermore, structural analysis of the peptides by circular dichroism and proton NMR has been performed for the Ov-2 series and Ov-3. These studies have confirmed the strong helical nature of Ov-2 and 3 in a lipid environment and the disruption of the helix of Ov-2(T7) and Ov-2(G10) (data not shown). Unlike the structural constraints required for the anti-microbial activity of these peptides, correlation of the structural analysis of the peptides with preliminary viricidal findings suggests that peptide changes that impart higher α-helicity and hydrophobic moment to the peptides enhance virucidal activity, whereas the net positive charge of the peptide does not appear to influence the viricidal activity.

The physical characteristics of theta defensins are dependent upon their circularization. In the noncircularized form, the peptide's bactericidal activity is salt dependent with high concentrations of NaCl inhibiting the defensin activity (Tang et al., 1999). Detailed physical characterization of the peptide structure on the antiviral activity of the theta defensins has not been performed.

### EXAMPLE 3

### Results

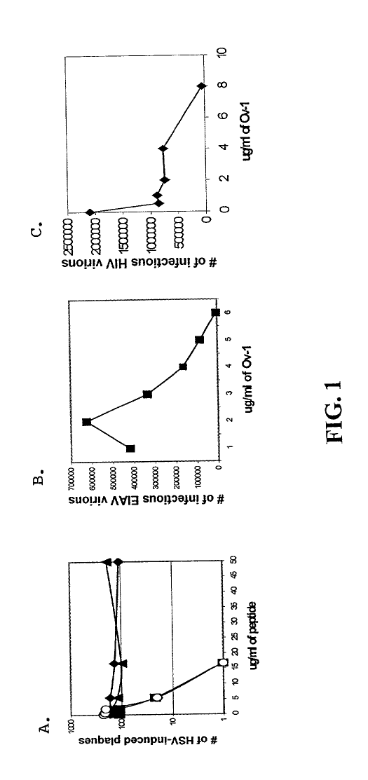

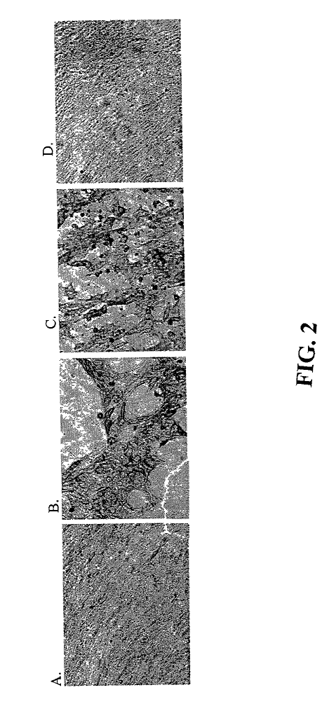

Initial studies on the ovispirins have investigated their anti-viral activity against herpes simplex 1 and 2 (HSV-1 and HSV-2), cytomegalovirus (CMV) and two retroviruses, human immunodeficiency virus (HIV) and equine infectious anemia virus (EIAV). The peptides were tested for anti-viral activity in tissue culture cells by preincubating virus stocks with peptide followed by addition of the mixture to cells. Readout for inhibition of viral infectivity was performed several different ways: the reduction of the number of herpes virus-induced plaques at 16 h (HSV) or 14 days (CMV), the number of EIAV or HIV antigen-expressing cells at 40 h, post infection, and the amount of HIV p24 antigen in the supernatants of infected cultures. Regardless of the assay used, anti-viral activities of Ov-1 and Ov-2 were detected. HSV-1 and -2 plaque formation was inhibited 3-6 fold at 6 μg/ml and approximately 100 fold at 18 μg/ml of Ov-1 (SEQ ID NO: 8, FIG. 1A). The synthetic peptide Ov-1 was the most effective in its anti-retroviral activity; both EIAV and HIV infectivity were decreased greater than 100 fold at 6-8 μg/ml (FIG. 1B and 1C). Similar concentrations of Ov-1 had a more modest effect (<10 fold) on the infectivity of adenovirus, a non-enveloped virus (data not shown). Studies with CMV were more qualitative, but effectively demonstrated that Ov-1 reduced plaque formation and the concentrations of Ov-1 that inhibited plaque formation had little to no effect on the monolayer of primary human fibroblasts (FIG. 2).

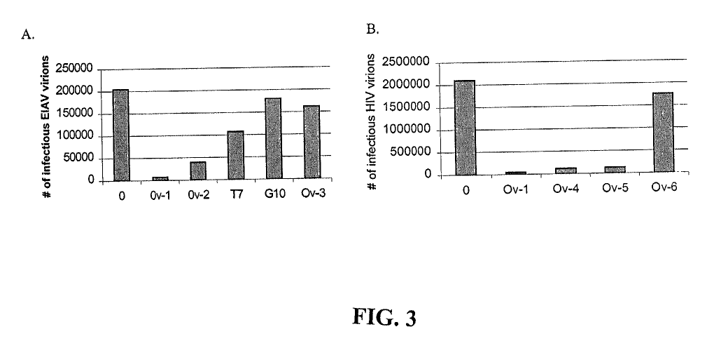

A peptide corresponding to the amino-terminal 18-amino acids of Ov-1, designated Ov-2 (SEQ ID NO: 12), was also an effective anti-retroviral agent decreasing EIAV infectious titers by more than 90% (FIG. 3). Similar to Ov-1, Ov-2 has high α-helicity in trifluorethanol (TFE) and a large hydrophobic moment. A 14-amino acid derivative, Ov-3 (SEQ ID NO: 13), had no effect over a wide range of concentrations. Interestingly, an α-helix of 18 to 20 amino acids is known to be required to span an eukaryotic membrane. The absence of anti-viral activity of Ov-3 is due to the inability of this peptide to span the viral lipid envelope. Eighteen amino acid forms that have decreased abilities to form a-helical structures in TFE due to amino acid substitutions had marked decreases in their anti-viral activity. These last findings show that the α-helical structure is critical for the anti-viral activity.

Preliminary studies with peptides corresponding to carboxy-terminal sequences of Ov-1 and HIV indicate that several, including Ov-4 and 5 (SEQ ID NOS: 17, 21), also have anti-retroviral activity (FIG. 3). Ov-6 (SEQ ID NO: 22) that corresponds to the 18 carboxyl-terminal amino acids of Ov-1 did not have appreciable anti-viral activity.

Studies with EIAV investigating the mode of action of the Ov class antivirals indicate that the peptides are acting early within the viral life cycle, perhaps acting on the viral particle itself. Ten μg/ml of Ov-1 was added to virus stocks of MA-1 either before, at the time of infection or various times following infection. Virus and peptide was then removed from the media 48 h post infection and the infected cultures were maintained for an additional 5 days to allow spread of any virus that is present within the culture. Monolayers were then immunostained for EIAV antigen expression. As shown in Table 6, addition of Ov-1 during the preincubation or at the time of infection was 100% effective in inhibiting virus replication; no viral antigen staining was observed in these cultures. Addition of the peptide 30 minutes or later following virus infection resulted in infection and spread of the virus throughout the monolayer.

TABLE 6
Time course of the inhibitory activity of Ov-1 against EIAV

| Time of peptide addition | Virus antigen positivity of culture |
|---|---|
| 30 m preaddition | − |
| 15 m preaddition | − |
| simultaneously | − |
| 30 m post addition | + |
| 60 m post addition | + |
| 90 m post addition | + |

These findings indicate that the peptides are acting at very early steps in the retroviral life cycle, perhaps before viral entry into the cell. This experiment in no way distinguishes whether the peptide is acting directly on the viral particle or somehow preventing virus attachment and/or entry. However, the ability of the peptide to inhibit both herpes virus infection and retroviral infections shows a broad spectrum mode of action of the peptide. Thus, the inventors have not predicted that Ov-1 is inhibiting specific cellular receptor attachments (such as gp120 interaction with CD4 and the chemokine receptors). Instead, they show that either the virion particle is disrupted by the peptide or the fusion event between the virion and the cell is disrupted. Disruption of the virion membrane would be most consistent with the known anti-microbial activity of natural cathelicidins.

Limited toxicity and immunogenicity studies of the Ov series of peptides have been performed. Results on the toxicity of the peptides in tissue culture show that peptide concentrations of 25-50 μg/ml are deleterious to the monolayer. In mice, moderate systemic doses of 5 mg/kg of Ov-1, Ov-2 or Ov-3 were found to have no adverse effects. Evidence of low immunogenicity and cytoxicity of Ov-2 has come from instillation studies into the lungs of mice. No imflammation, cytokine elevation or increase in blood markers was detected following the instillation of 100 μg of this peptide.

Synthetic theta defensins were tested for their abilities to inhibit HIV replication. Peptides were preincubated for 15-30 minutes with a known infectious dose of HIV. The mixture was added to HeLa cells that have been modified to permit HIV infection. Cells were maintained for 40 h, fixed and immunostained for HIV antigens. Numbers of antigen positive cells were counted within the wells. Addition of the theta defensins significantly decreased the numbers of HIV antigen positive cells. HTD-1 and RTD-3 had the greatest anti-viral activity. The anti-viral efficacy of oxidized and oxidized, circularized forms of HTD-1 and RTD-3 were tested. For both defensins, the oxidized, circularized form had greater anti-viral activity. Dose response curves using HTD-1 demonstrated that the IC₅₀ was enhanced about 10 fold by circularization with values of 0.48 ug/ml and 4.5 ug/ml for oxidized, circularized HTD-1 and oxidized HTD-1 respectively. Interestingly, RTD-1 an HDT-1 had no effect on the EIAV virus titer.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IV. References

The following references, to the extent that they provide exemplary procedural or other details supplementary to those set forth herein, are specifically incorporated herein by reference.

EPO 0273085

U.S. Pat. No. 4,554,101

Baichwal and Sugden, In: Gene Transfer, Kucherlapati R, ed., New York, Plenum Press, 117-148, 1986.

Bangham et al., J. Mol. Biol., 13: 238-252, 1965.

Benvenisty and Neshif, Proc. Nat'l Acad. Sci. USA, 83:9551-9555, 1986.

Capaldi et al., Biochem Biophys Res Commun, 74(2):425-33, 1977.

Chen and Okayama, Mol. Cell Biol., 7:2745-2752, 1987.

Deamer and Uster, “Liposome Preparation: Methods and Mechanisms,” In: Liposomes, M. Ostro (Ed.), 1983

Dubensky et al., Proc. Nat'l Acad. Sci. USA, 81:7529-7533, 1984.

Fechheimer et al., Proc. Nat'l. Acad. Sci. USA, 84:8463-8467, 1987.

Ferkol et al., FASEB J., 7:1081-1091, 1993.

Fraley et al., Proc. Nat'l Acad. Sci. USA, 76:3348-3352, 1979.

Freshner, In: Animal Cell Culture: a Practical Approach Second Edition, Oxford/N.Y., IRL Press, Oxford University Press, 1992.

Gallo et al., J. Biol. Chem., 272:13088-13093, 1997.

Ganz and Lehrer, Current Opinions Immun,. 10:41, 1998.

Ghosh and Bachhawat, In: Wu G. and C. Wu (eds.) Liver diseases, targeted diagnosis and therapy using specific receptors and ligands, New York: Marcel Dekker, pp. 87-104, 1991.

Ghosh-Choudhury et al, EMBO J, 6:1733-1739, 1987.

Gomez-Foix et al., J. Biol. Chem., 267:25129-25134, 1992.

Gopal, Mol. Cell Biol., 5:1188-1190, 1985.

Graham and Prevec, Biotechnology, 20:363-390, 1992.

Graham and Prevec, In: E. J. Murray (ed.), Methods in Molecular Biology: Gene Transfer and Expression Protocol, Clifton, N.J.: Humana Press, 7:109-128, 1991.

Graham and Van Der Eb, Virology, 52:456-467, 1973.

Graham et al., J. Gen. Virol., 36:59-72, 1977.

Gregoriadis (ed.), In: Drug Carriers in Biology and Medicine, pp 287-341, 1979.

Grunhaus and Horwitz, Seminar in Virology, 3:237-252, 1992.

Harland and Weintraub, J. Cell Biol., 101:1094-1099, 1985.

Hwang et al., Biochem. Cell Biol., 76(2-3):235-46, 1998.

Inouye et al., Nucleic Acids Res., 13:3101-3109, 1985.

Jones and Shenk, Cell, 13:181-188, 1978.

Kaneda, J. Biol Chem,.264(21):12126-9, 1989.

Karlsson et al., EMBO J., 5:2377-2385, 1986.

Kato et al., J. Biochem. Tokyo, 101:207-215, 1987.

Klein et al., Nature, 327:70-73, 1987.

Kyte J., Doolittle R. F., J. Mol. Biol, 157(1):105-32, 1982.

Levrero et al., Gene, 101:195-202,1991.

Macejak and Sarnow, Nature, 353:90-94, 1991.

Mulligan, Science, 260:926-932, 1993.

Nicolas and Rubenstein, In: Vectors: A survey of molecular cloning vectors and their uses, Rodriguez and Denhardt (eds.), Stoneham: Butterworth, pp. 494-513, 1988.

Nicolau and Sene, Biochem. Biophys. Acta, 721:185-190, 1982.

Nicolau et al., Methods Enzymol., 149:157-176, 1987.

Oren et al., Biopolymers, 47(6):451-63, 1998.

Pelletier and Sonenberg, Nature, 334:320-325, 1988.

Perales et al., Proc. Nat'l Acad. Sci., 91:4086-4090, 1994.

Potter etal., Proc. Nat'l Acad. Sci. USA, 81:7161-7165, 1984. Publications, Bern, Switzerland, pp. 555-67, 1981.

Racher et al., Biotechnology Techniques, 9:169-174, 1995.

Rich et al., Hum. Gene Ther., 4:461-476, 1993.

Ridgeway, In: Rodriguez R L, Denhardt D T, ed. Vectors: A survey of molecular cloning vectors and their uses, Stoneham: Butterworth, pp. 467-492, 1988.

Rippe et al., Mol Cell Biol., 10:689-695, 1990.

Russel et al., Infect Immun, 64(5):1565-8, 1996.

Stratford-Perricaudet and Perricaudet, In: Human Gene Transfer, Eds, 0. Cohen-Haguenauer and M. Boiron, Editions John Libbey Eurotext, France, pp. 51-61, 1991.

Stratford-Perricaudet et al, Hum. Gene Ther., 1:241-256, 1990.

Szoka and Papahadjopoulos, Proc. Nat'l Acad. Sci. USA, 75: 4194-4198, 1978.

Tang et al., Science, 286: 498-502, 1999.

Temin, In: Gene Transfer, Kucherlapati (ed.), New York: Plenum Press, pp. 149-188, 1986.

Top et al., J. Infect. Dis., 124:155-160, 1971.

Tran et al., J. Biol. Chem., 23:23, 2001.

Tur-Kaspa et al., Mol Cell Biol., 6:716-718, 1986.

Wagner et al., Science, 260:1510-1513, 1990.

Wu & Wu, Biochemistry, 27:887-892,1988.

Wu & Wu, J. Biol. Chem., 262:4429-4432, 1987.

Wu and Wu, Adv. Drug Delivery Rev., 12:159-167, 1993.

Yang et al., Proc. Nat'l Acad. Sci USA, 87:9568-9572, 1990

Zelenin et al., FEBS Lett., 280:94-96, 1991.

What is claimed is:

1. A method for reducing the infectivity of a virus comprising contacting said virus with a first anti-viral peptide, said peptide comprising a theta defensin peptide or amphipathic alpha helical structure in a lipid environment.

2. The method of claim 1, wherein said first anti-viral peptide is a naturally-occurring peptide.

3. The method of claim 2, wherein said naturally-occurring peptide is a cathelicidin.

4. The method of claim 3, wherein said cathelicidin is selected from the group consisting of a mouse cathelicidin, a monkey cathelicidin, a human cathelicidin, and a sheep cathelicidin.

5. The method of claim 1, wherein said first anti-viral peptide is a non-naturally occurring peptide.

6. The method of claim 1, wherein said peptide is about 13 to about 35 residues in length.

7. The method of claim 5, wherein said peptide contains a non-naturally occurring amino acid.

8. The method of claim 1, wherein the virus is an enveloped virus.

9. The method of claim 1, wherein the virus infects humans and is selected from the group consisting of HIV, HSV-1, HSV-2, EBV, varicella zoster virus, CMV, herpesvirus B, HHV6, HHV8, respiratory syncytial virus (RSV), influenza A, B and C viruses, hepatitis A, hepatitis B, hepatitis C, hepatitis G, smallpox, vaccinia virus, Marburg virus, ebola virus, dengue virus, West Nile virus, hantavirus, measles virus, mumps virus, rubella virus, rabies virus, yellow fever virus, Japanese encephalitis virus, Murray Valley encephalitis virus, Rocio virus, tick-borne encephalitis virus, St. Louis encephalitis virus, chikungynya virus, o'nyong-nyong virus, Ross River virus, Mayaro virus, human coronaviruses 229-E and OC43, vesicular stomatitis virus, sandfly fever virus, Rift Valley River virus, Lasa virus, lymphocytic choriomeningitis virus, Machupo virus, Junin virus, HTLV-I and -II.

10. The method of claim 1, wherein the virus infects sheep and is selected from the group consisting of border disease virus, Maedi virus, and visna virus.

11. The method of claim 1, wherein the virus infects cattle and is selected from the group consisting of bovine leukemia virus, bovine diarrhea virus, bovine lentivirus, and infectious bovine rhinotracheitis virus.

12. The method of claim 1, wherein the virus infects swine and is selected from the group consisting of swinepox, African swine fever virus, hemagluttinating virus of swine, hog cholera virus, and pseudorabies virus.

13. The method of claim 1, wherein the virus infects horses and is selected from the group consisting of bovine leukemia virus, bovine diarrhea virus, bovine lentivirus, and infectious bovine rhinotracheitis virus.

14. The method of claim 1, wherein the virus infects cats and is selected from the group consisting of feline immunodeficiency virus, feline leukemia virus, and feline infectious peritonitis virus.

15. The method of claim 1, wherein the virus infects fowl and is selected from the group consisting of Marek's disease virus, turkey bluecomb virus, infectious bronchitis virus of fowl, avian reticuloendotheliosis, sarcoma and leukemia viruses.

16. The method of claim 2, wherein the naturally-occurring peptide is selected from the group consisting of SEQ ID NOS: 1, 2, 3, 4, 5, 6 and 7.

17. The method of claim 5, wherein the non-naturally-occurring peptide is selected from the group consisting of SEQ ID NOS: 8, 9, 10, 11, 12, 13, 14, 15, 16, 17, 18, 19, 20, 21, 22, 23 and 24.

18. The method of claim 1, further comprising contacting said virus with a second anti-viral agent.

19. The method of claim 18, wherein said second anti-viral agent is a second anti-viral peptide distinct from said first anti-viral peptide.

20. The method of claim 18, wherein said second anti-viral agent is non-peptide pharmaceutical agent.

21. The method of claim 20, wherein said non-peptide pharmaceutical agent is selected from the group consisting of a protease inhibitor, a nucleoside analog, a viral polymerase inhibitor, and a viral integrase inhibitor.

22. The method of claim 1, wherein said first anti-viral peptide is contacted with said virus at a concentration of about 0.1 to about 50 μg per ml.

23. The method of claim 22, wherein said first anti-viral peptide is contacted with said virus at a concentration of about 1 to about 25 μg per ml.

24. The method of claim 23, wherein said first anti-viral peptide is contacted with said virus at a concentration of about 3 to about 10 μg per ml.

25. The method of claim 1, wherein said virus is located in a tissue or fluid sample.

26. The method of claim 25, wherein said tissue or fluid sample is selected from the group of whole blood, platelets, plasma, and packed blood cells.

27. The method of claim 1, wherein said virus is located in a living subject.

28. The method of claim 27, wherein said first anti-viral peptide is administered topically.

29. The method of claim 27, wherein said first anti-viral peptide is administered to a body cavity.

30. The method of claim 27, wherein said first anti-viral peptide is administered to a mucosal membrane.

31. The method of claim 27, wherein said first anti-viral peptide is administered by injection.

32. The method of claim 27, wherein said first anti-viral peptide is administered by inhalation.

33. The method of claim 27, wherein said first anti-viral peptide is administered orally.

34. The method of claim 27, wherein said first anti-viral peptide is administered to a wound site.

35. The method of claim 27, wherein said patient is immunosuppressed.

36. The method of claim 27, wherein said subject is not infected with said virus, and first anti-viral peptide is administered prior to the virus contacting the subject.

37. The method of claim 27, wherein said first anti-viral peptide is administered subsequent to the virus contacting the subject.

38. The method of claim 37, wherein said subject is chronically infected with said virus.

39. The method of claim 37, wherein said subject is latently infected with said virus.

40. The method of claim 37, wherein said subject is acutely infected with said virus.

41. An anti-viral composition comprising a first anti-viral peptide, said peptide comprising an amphipathic alpha helical structure or a theta defensin peptide in a lipid environment, and a second anti-viral agent.

42. The composition of claim 41, wherein said second anti-viral agent is a second anti-viral peptide distinct from said first anti-viral peptide.

43. The composition of claim 41, wherein said second anti-viral agent is a non-peptide pharmaceutical agent.

44. The composition of claim 43, wherein said non-peptide pharmaceutical agent is selected from the group consisting of a protease inhibitor, a nucleoside analog, a viral polymerase inhibitor, and a viral integrase inhibitor.

45. The composition of claim 41, formulated for topical administration.

46. The composition of claim 41, formulated for inhalation.

47. The composition of claim 41, formulated for administration to a mucosal membrane.

48. The composition of claim 41, wherein said composition is located in a sterile i.v. bag.

49. The composition of claim 41, wherein said composition is located in a sterile syringe.

50. The composition of claim 41, wherein said composition is located in sterile tubing.

51. An anti-viral composition comprising a first anti-viral peptide, said peptide comprising an amphipathic alpha helical structure in a lipid environment or a theta defensin peptide, and a contraceptive agent.

52. The composition of claim 51, wherein said composition is located in a condom.

53. The composition of claim 51, wherein said composition is formulated for use in a diaphragm.

54. The composition of claim 51, wherein said composition is formulated for intra-vaginal administration.

55. The composition of claim 51, wherein said contraceptive agent is spermicidal agent or a sperm anti-motility agent.

56. A method of rendering a virus-contaminated tissue or fluid sample safe for use comprising contacting said fluid sample with a first anti-viral peptide, said peptide comprising an amphipathic alpha helical structure in a lipid environment or a theta defensin peptide.

57. A method for reducing the number of infectious virus particles in a population of viruses comprising contacting said virus population with a first anti-viral peptide, said peptide comprising an amphipathic alpha helical structure in a lipid environment or a theta defensin peptide.

58. A method of protecting a subject from viral infection comprising administering to said subject a first anti-viral peptide, said peptide comprising an amphipathic alpha helical structure in a lipid environment or a theta defensin peptide.

59. A method for treating a subject with a viral infection comprising administering to said subject a first anti-viral peptide, said peptide comprising an amphipathic alpha helical structure in a lipid environment or a theta defensin peptide.

60. A method for preventing a recurrent viral infection in a subject harboring a latent virus comprising administering to said subject a first anti-viral peptide, said peptide comprising an amphipathic alpha helical structure in a lipid environment or a theta defensin peptide.

61. A method for controlling virus spread within a virally-infected subject comprising administering to said subject a first anti-viral peptide, said peptide comprising an amphipathic alpha helical structure in a lipid environment or a theta defensin peptide.

62. A method for reducing viral burden in a virally-infected subject comprising administering to said subject a first anti-viral peptide, said peptide comprising an amphipathic alpha helical structure in a lipid environment or a theta defensin peptide.

63. A method for reducing virus shed from a virally-infected subject comprising administering to said subject a first anti-viral peptide, said peptide comprising an amphipathic alpha helical structure in a lipid environment or a theta defensin peptide.

64. A method for reducing the percentage of virally-infected subjects in a population comprising administering to said population, regardless of viral infection status, a first anti-viral peptide, said peptide comprising an amphipathic alpha helical structure in a lipid environment or a theta defensin peptide.

65. A method of inducing latency in a virally-infected subject comprising administering to said subject a first anti-viral peptide, said peptide comprising an amphipathic alpha helical structure in a lipid environment or a theta defensin peptide.

66. The method of claim 1, wherein said first anti-viral peptide is encoded by a nucleic acid that is contained in an expression construct under the control of a promoter active in eukaryotic cells, wherein said expression construct is delivered into a host cell, and said cell supports production and secretion of said first anti-viral peptide which contacts said virus.

67. The method of claim 66, wherein said expression construct is an adenovirus.

68. The method of claim 66, wherein said host cell is infected by said virus.

69. The method of claim 66, wherein said nucleic acid further encodes an intracellular targeting signal fused to said first anti-viral peptide.

70. The method of claim 69, wherein said intracellular targeting signal targets said peptide to one or more of the endoplasmic reticulum, the Golgi apparatus and/or the cell surface.
